(19) United States
(12) Patent Application Publication
(10) Pub. No.: US 2025/0331585 A1
(43) Pub. Date: Oct. 30, 2025

(54) DISPOSABLE GLOVES

(71) Applicant: Jessica K. Freas, Glenview, IL (US)

(72) Inventor: Jessica K. Freas, Glenview, IL (US)

(21) Appl. No.: 19/188,644

(22) Filed: Apr. 24, 2025

Related U.S. Application Data
(60) Provisional application No. 63/638,370, filed on Apr. 24, 2024.

(51) Int. Cl. A41D 19/015; A41D 19/00; A61L 31/04

(52) U.S. Cl. CPC A41D 19/015; A41D 19/0044; A41D 19/0075; A61L 31/049

(57) ABSTRACT

Gloves configured to contain contaminants present on exterior surfaces of the glove. The glove includes a hand portion, a cuff portion adjoining the hand portion, a forearm portion adjoining and extending from the cuff portion, and a rim portion at a distal end of the forearm portion and defining an opening in the forearm portion for insertion of the wearer's hand into the glove. When the glove is worn by the wearer, the hand portion covers the wearer's hand, the cuff portion surrounds the wearer's wrist, and the forearm portion surrounds at least some of the wearer's forearm. The forearm portion has a bulbous shape that creates an annular-shaped cavity between the wearer's forearm and interior surfaces of the forearm portion, and the bulbous shape of the forearm portion defines a sufficient interior volume between the cuff portion and the rim portion to entirely contain the hand portion.

### CROSS-REFERENCE TO RELATED APPLICATIONS

This application claims the benefit of provisional U.S. Patent Application No. 63/638,370 filed Apr. 24, 2024, the contents of which are incorporated herein by reference.

### BACKGROUND OF THE INVENTION

The present invention generally relates to disposable gloves. The invention particularly relates to disposable gloves having an enlarged forearm portion configured to promote the ability to contain contaminants present on exterior surfaces of the glove as the glove is being removed from a wearer's hand.

Disposable gloves having an impermeable and typically elastic polymeric shell are commonly used to provide protection from physical, thermal, and/or chemical hazards. Such gloves are commonly categorized as general-purpose, examination-grade, or high-risk gloves. General-purpose disposable gloves are commonly used in a wide variety of industries, including automobile, food handling, general, hospitality and retail. Examination-grade disposable gloves are often used in the medical, dental, veterinary, law enforcement and childcare industries. In the United States, examination-grade medical gloves are regulated by the Food and Drug Administration (FDA) and are required to meet certain Acceptable Quality Level (AQL) standards related to being free of pinholes and providing certain protective properties. High-risk disposable gloves are generally associated with emergency medical services (EMS) and laboratory applications, such as situations that have a potential for exposure to blood, bodily fluids, infectious agents, chemicals, or disinfectants. General-purpose and examination-grade disposable gloves typically range from about 4 to 8 millimeters in thickness, though sometimes at greater thickness of about 8 to 10 millimeters in thickness, whereas high-risk disposable gloves commonly have thicknesses of about 10 millimeters or greater. In each case, the exterior surfaces of a disposable glove can become contaminated with what will be referred to herein as contaminants, which as used herein is meant to encompass all types of matter the nature of which will depend on the particular use of the glove.

Disposable gloves are generally intended to be used once (i.e., single-use) or for a limited number of uses (i.e., limited-use) and then disposed of and replaced. Certain limited-use disposable gloves may be categorized as “reusable” for some applications, such as gloves used to wash dishes, in which case a glove can be used as many times as its structural integrity will allow, for example, until pinholes, tears, or other defects form that would necessitate replacement. Single-use and limited-use gloves are often used not only to protect the wearer, but also to protect objects and surfaces touched by the wearer for the purpose of reducing the likelihood of contaminating the objects and surfaces. To proactively reduce the spread of contaminants, particularly infectious hazardous agents, disposable gloves are usually considered to be contaminated or soiled after a designated number of uses or upon completion of a specific activity, at which time it is essential to minimize contaminants present on exterior surfaces of the glove from being dispersed or otherwise transferred to the wearer and other objects in the surrounding environment as the glove is being removed from the wearer's hand.

Removal of disposable gloves may be difficult in certain situations, especially when time is a factor. For example, form-fitting gloves and/or gloves having certain internal surface properties (e.g., tackiness) may hinder the wearer's ability to quickly remove the glove. In addition to being inconvenient, difficulty in removing disposable gloves may pose a serious threat to the wearer and/or others. For example, medical personnel may be required to quickly remove and/or replace a pair of medical gloves prior to or during a medical emergency and any delays may reduce the likelihood of successful treatment of a patient. As another example, a laboratory researcher may have a harmful chemical spill on the glove and be required to quickly remove the glove prior to the chemical potentially penetrating the polymeric shell. In both of these examples, any delay in glove removal may have life-threatening consequences, and yet removal of the glove from the wearer's hand must be performed carefully to minimize contaminants present on exterior surfaces of the glove from being dispersed or otherwise transferred to the wearer and other objects in the surrounding environment.

In view of the above, it can be appreciated that there are certain problems, shortcomings or disadvantages associated with disposable gloves, and that it would be desirable if disposable gloves were available that were constructed in a manner that promoted ease of removal while also minimizing the dispersal of contaminants present on exterior surfaces of the glove.

### BRIEF DESCRIPTION OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides gloves that promote the ability to contain contaminants present on exterior surfaces of the glove as the glove is being removed from a wearer's hand.

According to a nonlimiting aspect of the invention, a glove is provided that is formed of a polymeric material and configured to be donned on a hand of a wearer. The glove includes a hand portion, a cuff portion adjoining the hand portion, a forearm portion adjoining and extending from the cuff portion, and a rim portion at a distal end of the forearm portion and defining an opening in the forearm portion for insertion of the hand of the wearer into the glove. When the glove is worn by the wearer, the hand portion is configured to cover the wearer's hand including a palm and fingers thereof, the cuff portion is configured to surround a wrist of the wearer, the forearm portion is configured to surround at least some of a forearm of the wearer and has a bulbous shape that creates an annular-shaped cavity between the wearer's forearm and interior surfaces of the forearm portion, and the bulbous shape of the forearm portion defines a sufficient interior volume between the cuff portion and the rim portion to entirely contain the hand portion.

According to another nonlimiting aspect of the invention, a method of using the glove is provided. The method includes donning the glove by inserting the hand of the wearer into the glove through the opening defined by the rim portion such that fingers and a thumb of the hand are within the hand portion, the wrist of the wearer is within the cuff portion, and at least a portion of the forearm of the wearer is within the forearm portion, and then performing an activity while wearing the glove. Thereafter, the hand portion of the glove is encased within the forearm portion of the glove by pulling the forearm portion toward and over the hand portion such that the interior surfaces of the forearm portion are exposed and the hand portion is entirely contained within the forearm portion between the cuff portion and the rim portion. The glove may then be removed from the hand of the wearer.

Technical effects of a glove as described above preferably include the ability to quickly remove the glove from a wearer's hand and simultaneously contain and encase within the forearm portion any contaminants that may be present on exterior surfaces of the glove, such as contaminants that result from using the glove.

Other aspects and advantages of this invention will be appreciated from the following detailed description.

### BRIEF DESCRIPTION OF THE DRAWINGS

FIG. 1 represents a disposable glove having a hand portion and a bulbous-shaped forearm portion in accordance with a nonlimiting embodiment of the present invention.

FIG. 2 schematically represents the hand portion of the glove of FIG. 1 entirely contained within the bulbous shape of the forearm portion after the forearm portion has been pulled toward and over the hand portion to result in the forearm portion being turned inside-out.

### DETAILED DESCRIPTION OF THE INVENTION

The intended purpose of the following detailed description of the invention and the phraseology and terminology employed therein is to describe what is shown in the drawings, which include the depiction of and/or relate to one or more nonlimiting embodiments of the invention, and to describe certain but not all aspects of the embodiment(s) to which the drawings relate. The following detailed description also identifies certain but not all alternatives of the embodiment(s) depicted in the drawings. As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended claims, and not the detailed description, are intended to particularly point out subject matter regarded as aspects of the invention, including certain but not necessarily all of the aspects and alternatives described in the detailed description.

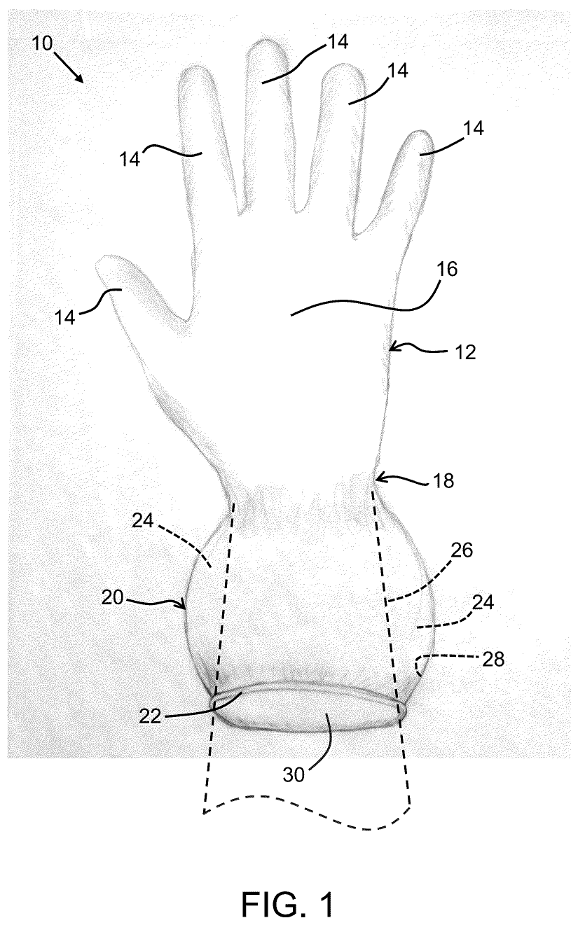

FIG. 1 represents a nonlimiting embodiment of a disposable glove 10 having an enlarged forearm portion 20 configured to promote ease of removal and also promote the ability to contain contaminants present on exterior surfaces of the glove 10. The glove 10 includes a hand portion 12 configured to surround and cover a wearer's hand including a central portion 16 configured to receive of the wearer's palm and sheaths 14 configured to receive the wearer's fingers and thumb. A cuff portion 18 adjoins the hand portion 12 and is configured to surround and cover the wearer's wrist. The enlarged forearm portion 20 adjoins and extends from the cuff portion 18 in a direction away from the hand portion 12, and is configured to cover at least some of the wearer's forearm 26 (e.g., at least that portion of the forearm adjacent the wrist). An edge or rim portion 22 is disposed at a distal end of the forearm portion 20 (i.e., opposite the hand portion 12) and defines an opening 30 for insertion of the wearer's hand into the glove 10.

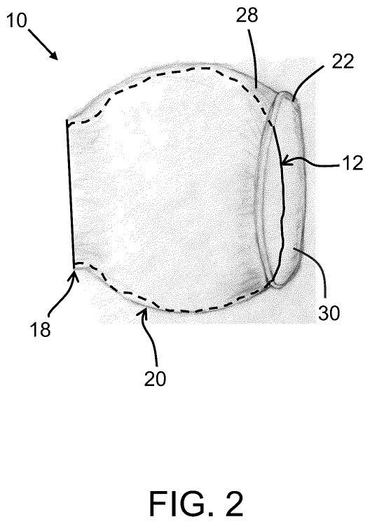

The hand portion 12 may be configured to be substantially form-fitting (conformal) to a wearer's hand to promote grip, dexterity, and tactile sensation of the wearer's hand. The cuff portion 18 may also configured to be substantially form-fitting to the wearer's wrist to reduce the likelihood that the wearer's hand will slip out of the hand portion 12 during certain activities. The rim portion 22 is preferably configured to be substantially form-fitting completely around the wearer's forearm 26 so as to form a seal around the wearer's forearm 26 that can serve to reduce the likelihood of slippage during use and/or reduce the likelihood of contaminants entering the interior of the glove 10 during use. To promote a seal, the rim portion 22 may be elastic or otherwise incorporate an elastic member that causes the rim portion 22 to be biased to elastically contract radially inward. In contrast, the forearm portion 20 is preferably configured to be substantially loose-fitting around the wearer's forearm 26. As represented in FIG. 1, the forearm portion 20 preferably has a bulbous, generally spherical shape that creates an annular-shaped void or cavity 24 between the wearer's forearm 26 and interior surfaces of the forearm portion 20 when the glove 10 is worn by the user (wearer). As represented in FIG. 1, the cavity 24 creates an airspace that surrounds the wearer's forearm 26 within the forearm portion 20. As will be discussed below, the forearm portion 20 is adapted for encasing and sealing any contaminants (including any biological matter, chemicals, or other matter) desired to be disposed of along with the glove 10 or, in the case of a reusable or washable glove, any contaminants that can be contained with the glove 10 and later disposed of at a more appropriate location and time. For this purpose, the bulbous shape of the forearm portion 20 has a sufficient interior volume between the cuff portion 18 and the rim portion 22 to entirely contain the hand portion 12, albeit in a crumpled state as schematically represented in FIG. 2.

The hand portion 12, cuff portion 18, and forearm portion 20 are integral regions of the disposable glove 10 that in combination preferably comprise or define an elastic liquid-impermeable polymeric shell. The glove 10 may be formed of various polymeric materials including but not limited to certain natural and synthetic rubbers. Specific examples may include (but are not limited to) latex, polyvinyl chloride, vinyl, nitrile rubber, or neoprene. These materials may allow the glove 10 to be cleaned and reused multiple times or limit the glove 10 to a single use. The glove 10 may be fabricated by various methods including those commonly used to produce existing disposable gloves. The disposable glove 10 may be configured for categorization as general-purpose, examination-grade, or high risk, may be sterile or nonsterile, and may be powdered or powder-free. For example, the glove 10 may include a corn starch powder on the interior surfaces thereof to lubricate the interior surfaces during donning, use, and removal of the glove 10. For embodiments in which the glove 10 is powder-free, the interior surfaces of the glove 10 may undergo certain chemical treatments (e.g., chlorination) to reduce the tackiness of the polymeric materials used to form the glove 10.

The polymeric shell of the glove 10 may be formed by a single layer or multiple layers. Additionally, the glove 10 may include a single layer or multiple layers that form the polymeric shell and one or more additional layers that may or may not contain a polymeric material. For example, in certain embodiments the glove 10 may include a liner that includes a woven or nonwoven fabric material adhered to internal surfaces of the glove 10. Such a liner may be configured, for example, to provide a thermal barrier, promote ease of donning and/or removing the glove 10, promote comfort, etc. The glove 10 may have smooth exterior surfaces and/or may include one or more areas having textured arrays of protuberances or recesses to promote grip.

FIG. 1 represents the bulbous shape of the forearm portion 20 of the glove 10 as causing the forearm portion 20 to extend radially outward relative to the cuff and rim portions 18 and 22 so as to have a larger cross-sectional area than the cuff portion 18 and the rim portion 22, form the annular cavity 24, and also provide an airspace between the wearer's forearm 26 and interior surfaces 28 of the forearm portion 20 while the glove 10 is worn by the wearer. The additional material of the forearm portion 20 enables the wearer to grasp the forearm portion 20 and/or rim portion 22 and pull the forearm portion 20 toward and over the hand portion 12 after use of the glove 10, such that the forearm portion 20 is turned inside-out and the hand portion 12 is entirely encased within the inverted forearm portion 20, such as schematically represented in FIG. 2. In this manner, any contaminants on the exterior surfaces of the hand and cuff portions 12 and 18 are also contained, encased, and optionally sealed by the rim portion 22 within the inverted forearm portion 20. For this purpose, and as previously noted, the bulbous shape of the forearm portion 20 defines a sufficient large interior volume to entirely contain at least the hand portion 12 (including each of its sheaths 14 and central portion 16) and optionally also the cuff portion 18. If the rim portion 22 is elastic as discussed previously, the opening 30 formed by the rim portion 22 is preferably closer in size to the cuff portion 18 than to the forearm portion 20 to enable the forearm portion 20 to better contain the hand portion 12 and any contaminants thereon.

After the forearm portion 20 is inverted to cover the remainder of the glove 10, the glove 10 may be quickly and easily removed from the wearer's hand, such as grasping the inverted cuff portion 18 (FIG. 2), thus promoting ease of removal of the glove 10. In this manner, the forearm portion 20 enables the glove 10 to be removed without touching those areas of the glove 10 (e.g., exterior surfaces of the hand and cuff portions 12 and 18) that were contaminated during use. Thereafter, the encased contaminants can be disposed of along with the glove 10 or, in the case that the glove 10 is intended to be washed and reused, the contaminants encased within the forearm 20 can be emptied and later disposed of at a more appropriate location or time. In view of its intended use, it should be appreciated that the forearm portion 20 is not limited to the specific shape and relative size represented in FIG. 1. Rather, the forearm portion 20 may have various other shapes and relative sizes configured to form an interior volume when inverted that may be used to encase at least the hand portion 12 of the glove 10. In addition, the forearm portion 20 may provide additional protection of the wearer's forearm 26 relative to existing disposable gloves that include a polymeric shell that extends only to the wearer's wrist.

In view of the above, the disposable glove 10 provides for a method of using the glove 10 that includes quickly and efficiently removing the glove 10 from a wearer's hand after the glove 10 has been used and/or soiled. Such a method includes donning the glove 10 by inserting the wearer's hand into the glove 10 through the opening 30 defined by the rim portion 22 such that the wearer's fingers and thumb are within the sheaths 14, the wearer's palm is within the central portion 16, the wearer's wrist is within the cuff portion 18, and at least a portion of the wearer's forearm 26 is within the forearm portion 20. The wearer may then perform one or more of various activities while wearing the glove 10, including those common for wearers of existing disposable rubber gloves and medical gloves (e.g., for surgical and medical procedures). After the task is completed and/or the glove 10 has become soiled, the hand portion 12 of the glove 10 can be encased within the forearm portion 20 by pulling the forearm portion 20 toward and over the hand portion 12 such that the forearm portion 20 is inverted inside-out over the hand portion 12 and the hand portion 12 is entirely contained within the forearm portion 20 between the cuff portion 18 and the rim portion 12, as schematically represented in FIG. 2. Thereafter, the glove 10 can be removed from the hand of the wearer. In this manner, the exterior surfaces of the forearm portion 20 are pulled toward and over the exterior surfaces of the hand portion 12 to an extent that the interior surfaces 28 of the forearm portion 20 are exposed instead of the exterior surfaces thereof. In such cases, the glove 10 is able to contain any contaminants present on its exterior surfaces, including the exterior surfaces of the hand, cuff, and forearm portions 12, 18, and 20. Optionally, the forearm portion 20 and/or rim portion 22 may be further pulled to an extent such that the hand portion 12 is also turned inside-out such that interior surfaces of the hand portion 12 are also exposed and contaminants present on its exterior surfaces are further contained and encased by the hand portion 12.

Benefits of a disposable glove such as represented in FIG. 1 include the ability to contain and encase materials commonly handled with disposable gloves when performing certain tasks, including various liquids and solids, by inverting the forearm portion 20 over and around the hand portion 12 of the glove 10 to create a sealed cavity around the hand portion 12 as schematically represented in FIG. 2. This capability is helpful in situations where a localized job can be performed while keeping the wearer's hands clean while simultaneously providing a way to contain contaminants encountered when performing the task. For example, a glove of the type shown in FIG. 1 can be particularly useful when field dressing in locations where a sink is not readily available, or when changing a diaper or cleaning up vomit or blood in locations where soap and water are not readily available, such as in public settings (such as an airport) or outdoors. The wearer of the glove can perform a task, retain the cleanliness of their hands within the glove, then proceed to encase contaminants encountered during the task. The entire glove can then be removed and disposed of, or in the case of glove that can be safely reused, the glove can be cleaned of contaminants after the forearm portion 20 has been returned to its state as shown in FIG. 1.

As previously noted above, though the foregoing detailed description describes certain aspects of one or more particular embodiments of the invention, alternatives could be adopted by one skilled in the art. As such, and again as was previously noted, it should be understood that the invention is not necessarily limited to any particular embodiment described herein or illustrated in the drawings.

1. A glove formed of a polymeric material and configured to be donned on a hand of a wearer, the glove comprising:
a hand portion;
a cuff portion adjoining the hand portion;
a forearm portion adjoining and extending from the cuff portion; and
a rim portion at a distal end of the forearm portion and defining an opening in the forearm portion for insertion of the hand of the wearer into the glove;
wherein, when worn by the wearer, the hand portion is configured to cover the wearer's hand including a palm and fingers thereof, the cuff portion is configured to surround a wrist of the wearer, the forearm portion is configured to surround at least some of a forearm of the wearer and has a bulbous shape that creates an annular-shaped cavity between the wearer's forearm and interior surfaces of the forearm portion, and the bulbous shape of the forearm portion defines a sufficient interior volume between the cuff portion and the rim portion to entirely contain the hand portion.

2. The glove of claim 1, wherein the forearm portion extends radially outward relative to the cuff portion and the rim portion so as to have a larger cross-sectional area than the cuff portion and the rim portion.

3. The glove of claim 1, wherein the bulbous shape of the forearm portion is spherical shaped.

4. The glove of claim 1, wherein the rim portion is elastic such that the rim portion is biased to elastically contract radially inward.

5. The glove of claim 1, wherein the opening of the rim portion is closer in size to the cuff portion than to the forearm portion.

6. The glove of claim 1, wherein the hand portion, the cuff portion, the forearm portion, and the rim portion are integral regions of the glove that define a liquid-impermeable polymeric shell.

7. The glove of claim 1, wherein interior surfaces of the glove have a powder material deposited thereon to promote lubrication thereof.

8. The glove of claim 1, wherein the glove has a chemical treatment that reduces a tackiness of the polymeric material.

9. The glove of claim 1, wherein the glove has multiple layers.

10. The glove of claim 1, wherein the polymeric material is latex, polyvinyl chloride, vinyl, nitrile rubber, or neoprene.

11. The glove of claim 1, wherein one or more exterior surfaces of the glove are textured.

12. The glove of claim 1, wherein the glove is a general-purpose glove.

13. The glove of claim 1, wherein the glove is an examination-grade glove.

14. The glove of claim 1, wherein the glove is a high-risk glove.

15. The glove of claim 1, wherein the glove is reusable.

16. The glove of claim 1, wherein the glove is disposable.

17. The glove of claim 1, wherein the glove is sterile.

18. A method of using the glove of claim 1, the method comprising:
donning the glove by inserting the hand of the wearer into the glove through the opening defined by the rim portion such that fingers and a thumb of the hand are within the hand portion, the wrist of the wearer is within the cuff portion, and at least a portion of the forearm of the wearer is within the forearm portion;
performing an activity while wearing the glove;
encasing the hand portion of the glove within the forearm portion of the glove by pulling the forearm portion toward and over the hand portion such that the interior surfaces of the forearm portion are exposed and the hand portion is entirely contained within the forearm portion between the cuff portion and the rim portion; and then
removing the glove from the hand of the wearer.

19. The method of claim 18, further comprising further pulling the forearm portion such that the hand portion is also turned inside-out such that interior surfaces of the hand portion are exposed.

20. The method of claim 18, wherein the activity is a surgical or a medical procedure.
